(19) United States
(12) Patent Application Publication
(10) Pub. No.: US 2023/0342922 A1
(43) Pub. Date: Oct. 26, 2023

(54) OPTIMIZING ULTRASOUND SETTINGS

(71) Applicant: EchoNous, Inc., Redmond, WA (US)

(72) Inventors: Babajide Ayinde, Redmond, WA (US); Matthew Cook, Redmond, WA (US); Soham Bhosale, Redmond, WA (US); Maya Keselman, Redmond, WA (US)

(21) Appl. No.: 18/054,458

(22) Filed: Nov. 10, 2022

Related U.S. Application Data
(60) Provisional application No. 63/333,953, filed on Apr. 22, 2022.

(51) Int. Cl. G06T 7/00; G06N 3/08

(52) U.S. Cl. CPC G06T 7/0012; G06N 3/08; G06T 2207/20081; G06T 2207/10132

(57) ABSTRACT

A machine learning model is described that usable to improve the quality of an ultrasound image captured from a person using a set of ultrasound machine setting values that is collectively sub-optimal. Different versions of the model predict from such a starting ultrasound image either (a) a new set of setting values that can be used to reimage the person to produce a higher-quality ultrasound image, or (b) this higher-quality ultrasound image directly.

### CROSS-REFERENCE TO RELATED APPLICATIONS

This application claims the benefit of provisional U.S. Application No. 63/333,953, filed Apr. 22, 2022 and entitled “OPTIMIZING ULTRASOUND SETTINGS,” which is hereby incorporated by reference in its entirety.

In cases where the present application conflicts with a document incorporated by reference, the present application controls.

### BACKGROUND

Ultrasound imaging is a useful medical imaging modality. For example, internal structures of a patient's body may be imaged before, during or after a therapeutic intervention. Also, qualitative and quantitative observations in an ultrasound image can be a basis for diagnosis. For example, ventricular volume determined via ultrasound is a basis for diagnosing, for example, ventricular systolic dysfunction and diastolic heart failure.

A healthcare professional typically holds a portable ultrasound probe, sometimes called a “transducer,” in proximity to the patient and moves the transducer as appropriate to visualize one or more target structures in a region of interest in the patient. A transducer may be placed on the surface of the body or, in some procedures, a transducer is inserted inside the patient's body. The healthcare professional coordinates the movement of the transducer so as to obtain a desired presentation on a screen, such as a two-dimensional cross-section of a three-dimensional volume.

Particular views of an organ or other tissue or body feature (such as fluids, bones, joints or the like) can be clinically significant. Such views may be prescribed by clinical standards as views that should be captured by the ultrasound operator, depending on the target organ, diagnostic purpose or the like.

It is common for ultrasound machines to operate in accordance with values set for one or more user settings of the machine. Typical machines permit users to set values for settings such as one or more of the following: Depth, Gain, Time-Gain-Compensation (“TGC”), Body Type, and Imaging Scenario. Depth specifies the distance into the patient the ultrasound image should reach. Time-Gain-Compensation specifies the degree to which received signal intensity should be increased with depth to reduce non-uniformity of image intensity resulting from tissue attenuation of the ultrasound signal. Body Type indicates the relative size of the patient's body. And Imaging Scenario specifies a region or region type of the body to be imaged, such as Heart, Lungs, Abdomen, or Musculoskeletal. In some embodiments, the facility uses the value specified for the Imaging Scenario setting as a basis for automatically specifying values for other common constituent settings, such as transmit wave form, transmit voltage, bandpass filter, apodization, compression, gain, persistence, smoothing/spatial filter, and speckle reduction.

### BRIEF DESCRIPTION OF THE DRAWINGS

FIG. 1 is a schematic illustration of a physiological sensing device, in accordance with one or more embodiments of the present disclosure.

FIG. 2 is a block diagram showing some of the components typically incorporated in at least some of the computer systems and other devices on which the facility operates.

FIG. 3 is a general data flow diagram showing the operation of the facility.

FIG. 4 is a general flow diagram showing the operation of the facility with respect to one or more machine learning models used by the facility.

FIG. 5 is a data flow diagram showing a process performed by the facility in some of the setting improvement embodiments.

FIG. 6 is a flow diagram showing a process performed by the facility in some of the setting improvement embodiments.

FIG. 7 is a model architecture diagram showing the organization of a model used by the facility in some of the setting improvement embodiments.

FIG. 8 is data flow diagram showing a process performed by the facility in some of the generative embodiments.

FIG. 9 is a flow diagram showing a process performed by the facility in some of the generative embodiments.

FIG. 10 is a model architecture diagram showing the organization of machine learning model used by the facility in some of the generative embodiments.

FIG. 11 is a flow diagram showing a process performed by the facility in some embodiments in order to train a machine learning model used by the facility, either a setting improvement model or a generative model.

### DETAILED DESCRIPTION

The inventors have recognized that it is burdensome to require the operator of an ultrasound machine to adjust the value of user settings for each patient, and often for each of multiple imaging studies for the same patient. Further, it typically takes time for a new operator to learn how to choose the correct values for these settings; until s/he does, many studies may need to be repeated in order to obtain good quality results for them.

In response, the inventors have conceived and reduced to practice a software and/or hardware facility that automatically establishes values for ultrasound settings for an ultrasound study (“the facility”).

The facility acquires an initial image using a set of initial setting values. In various embodiments, these initial setting values are default setting values that are the same for every study; user-specified setting values; and/or setting values automatically determined based upon inputs about the user, such body type setting values determined using photographic images, electronic medical record fields for the patient specifying body type, body mass index, or weight, etc.

In some “setting improvement” embodiments, the facility uses a setting value evaluation machine learning model to discern from the initial image whether the initial value of each setting was optimal. The facility automatically adjusts the setting values that it determines were not optimal to be more optimal for reimaging of the patient. In some embodiments, the facility uses the same setting value evaluation model to evaluate one or more subsequent images for setting value optimality, and continues to adjust those settings whose values are still not optimal.

In some embodiments, the facility trains the setting value evaluation model using training observations generated based on sets of images captured from each training subject in a group of training subjects. The group of training subjects is constituted in a way designed to cover the range of possible values of any patient-specific settings, such as body type. For each subject, the facility captures a number of ultrasound images using sets of setting values selected from the n-dimensional volume in which each dimension represents the range of possible values for a different setting. In some cases, the images captured for each patient collectively cover a set of organs, or imaging sites or scenarios of other types. For each subject, for each imaging site, the facility solicits a human expert to select the one of these images that is of the highest-quality. The facility uses the selection of the highest quality image for each combination of subject and imaging site to construct a training observation for each image that it uses to train the setting value evaluation model, where the independent variable is the image, and the dependent variables are the setting values are the setting values of the image of the same subject and site that was selected as highest-quality.

In some “generative” embodiments, the facility applies a generative model to transform the initial image into an improved image whose level of quality is higher. In some embodiments, the generative model is a conditional generative adversarial network, or “cGAN.” In some embodiments, the facility trains the generative network using training observations it constructs from the images captured and selected as described above. In particular, the training observation generated by the facility for each image has as its independent variable the captured image, and has as its dependent variable the image selected as highest-quality for the same combination of subject and site.

By performing in some or all of these ways, the facility reduces the levels of needed operator skill and experience, time, and inaccuracy incurred by ultrasound studies.

Additionally, the facility improves the functioning of computer or other hardware, such as by reducing the dynamic display area, processing, storage, and/or data transmission resources needed to perform a certain task, thereby enabling the task to be permitted by less capable, capacious, and/or expensive hardware devices, and/or be performed with lesser latency, and/or preserving more of the conserved resources for use in performing other tasks. For example, by reducing the amount of time for which the ultrasound machine is used for a particular study, the ultrasound machine can be used for a great number of studies during its lifetime, or a version that can be used for the same number of studies while being manufactured at lower cost. Also, by reducing the number of unsuccessful studies that must be repeated, the facility increases the availability of ultrasound machines for additional original studies.

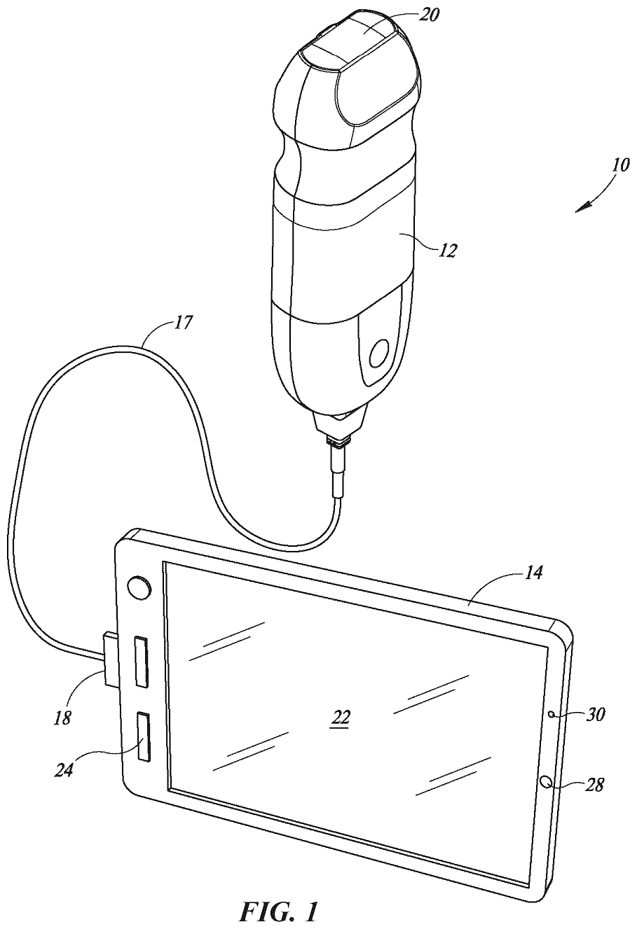

FIG. 1 is a schematic illustration of a physiological sensing device 10, in accordance with one or more embodiments of the present disclosure. The device 10 includes a probe 12 that, in the illustrated embodiment, is electrically coupled to a handheld computing device 14 by a cable 17. The cable 17 includes a connector 18 that detachably connects the probe 12 to the computing device 14. The handheld computing device 14 may be any portable computing device having a display, such as a tablet computer, a smartphone, or the like. In some embodiments, the probe 12 need not be electrically coupled to the handheld computing device 14, but may operate independently of the handheld computing device 14, and the probe 12 may communicate with the handheld computing device 14 via a wireless communication channel.

The probe 12 is configured to transmit an ultrasound signal toward a target structure and to receive echo signals returning from the target structure in response to transmission of the ultrasound signal. The probe 12 includes an ultrasound sensor 20 that, in various embodiments, may include an array of transducer elements (e.g., a transducer array) capable of transmitting an ultrasound signal and receiving subsequent echo signals.

The device 10 further includes processing circuitry and driving circuitry. In part, the processing circuitry controls the transmission of the ultrasound signal from the ultrasound sensor 20. The driving circuitry is operatively coupled to the ultrasound sensor 20 for driving the transmission of the ultrasound signal, e.g., in response to a control signal received from the processing circuitry. The driving circuitry and processor circuitry may be included in one or both of the probe 12 and the handheld computing device 14. The device 10 also includes a power supply that provides power to the driving circuitry for transmission of the ultrasound signal, for example, in a pulsed wave or a continuous wave mode of operation.

The ultrasound sensor 20 of the probe 12 may include one or more transmit transducer elements that transmit the ultrasound signal and one or more receive transducer elements that receive echo signals returning from a target structure in response to transmission of the ultrasound signal. In some embodiments, some or all of the transducer elements of the ultrasound sensor 20 may act as transmit transducer elements during a first period of time and as receive transducer elements during a second period of time that is different than the first period of time (i.e., the same transducer elements may be usable to transmit the ultrasound signal and to receive echo signals at different times).

The computing device 14 shown in FIG. 1 includes a display screen 22 and a user interface 24. The display screen 22 may be a display incorporating any type of display technology including, but not limited to, LCD or LED display technology. The display screen 22 is used to display one or more images generated from echo data obtained from the echo signals received in response to transmission of an ultrasound signal, and in some embodiments, the display screen 22 may be used to display color flow image information, for example, as may be provided in a Color Doppler imaging (CDI) mode. Moreover, in some embodiments, the display screen 22 may be used to display audio waveforms, such as waveforms representative of an acquired or conditioned auscultation signal.

In some embodiments, the display screen 22 may be a touch screen capable of receiving input from an operator that touches the screen. In such embodiments, the user interface 24 may include a portion or the entire display screen 22, which is capable of receiving operator input via touch. In some embodiments, the user interface 24 may include one or more buttons, knobs, switches, and the like, capable of receiving input from an operator of the ultrasound device 10. In some embodiments, the user interface 24 may include a microphone 30 capable of receiving audible input, such as voice commands.

The computing device 14 may further include one or more audio speakers 28 that may be used to output acquired or conditioned auscultation signals, or audible representations of echo signals, blood flow during Doppler ultrasound imaging, or other features derived from operation of the device 10.

The probe 12 includes a housing, which forms an external portion of the probe 12. The housing includes a sensor portion located near a distal end of the housing, and a handle portion located between a proximal end and the distal end of the housing. The handle portion is proximally located with respect to the sensor portion.

The handle portion is a portion of the housing that is gripped by an operator to hold, control, and manipulate the probe 12 during use. The handle portion may include gripping features, such as one or more detents, and in some embodiments, the handle portion may have a same general shape as portions of the housing that are distal to, or proximal to, the handle portion.

The housing surrounds internal electronic components and/or circuitry of the probe 12, including, for example, electronics such as driving circuitry, processing circuitry, oscillators, beamforming circuitry, filtering circuitry, and the like. The housing may be formed to surround or at least partially surround externally located portions of the probe 12, such as a sensing surface. The housing may be a sealed housing, such that moisture, liquid or other fluids are prevented from entering the housing. The housing may be formed of any suitable materials, and in some embodiments, the housing is formed of a plastic material. The housing may be formed of a single piece (e.g., a single material that is molded surrounding the internal components) or may be formed of two or more pieces (e.g., upper and lower halves) which are bonded or otherwise attached to one another.

In some embodiments, the probe 12 includes a motion sensor. The motion sensor is operable to sense a motion of the probe 12. The motion sensor is included in or on the probe 12 and may include, for example, one or more accelerometers, magnetometers, or gyroscopes for sensing motion of the probe 12. For example, the motion sensor may be or include any of a piezoelectric, piezoresistive, or capacitive accelerometer capable of sensing motion of the probe 12. In some embodiments, the motion sensor is a tri-axial motion sensor capable of sensing motion about any of three axes. In some embodiments, more than one motion sensor 16 is included in or on the probe 12. In some embodiments, the motion sensor includes at least one accelerometer and at least one gyroscope.

The motion sensor may be housed at least partially within the housing of the probe 12. In some embodiments, the motion sensor is positioned at or near the sensing surface of the probe 12. In some embodiments, the sensing surface is a surface which is operably brought into contact with a patient during an examination, such as for ultrasound imaging or auscultation sensing. The ultrasound sensor 20 and one or more auscultation sensors are positioned on, at, or near the sensing surface.

In some embodiments, the transducer array of the ultrasound sensor 20 is a one-dimensional (1D) array or a two-dimensional (2D) array of transducer elements. The transducer array may include piezoelectric ceramics, such as lead zirconate titanate (PZT), or may be based on microelectromechanical systems (MEMS). For example, in various embodiments, the ultrasound sensor 20 may include piezoelectric micromachined ultrasonic transducers (PMUT), which are microelectromechanical systems (MEMS)-based piezoelectric ultrasonic transducers, or the ultrasound sensor 20 may include capacitive micromachined ultrasound transducers (CMUT) in which the energy transduction is provided due to a change in capacitance.

The ultrasound sensor 20 may further include an ultrasound focusing lens, which may be positioned over the transducer array, and which may form a part of the sensing surface. The focusing lens may be any lens operable to focus a transmitted ultrasound beam from the transducer array toward a patient and/or to focus a reflected ultrasound beam from the patient to the transducer array. The ultrasound focusing lens may have a curved surface shape in some embodiments. The ultrasound focusing lens may have different shapes, depending on a desired application, e.g., a desired operating frequency, or the like. The ultrasound focusing lens may be formed of any suitable material, and in some embodiments, the ultrasound focusing lens is formed of a room-temperature-vulcanizing (RTV) rubber material.

In some embodiments, first and second membranes are positioned adjacent to opposite sides of the ultrasound sensor 20 and form a part of the sensing surface. The membranes may be formed of any suitable material, and in some embodiments, the membranes are formed of a room-temperature-vulcanizing (RTV) rubber material. In some embodiments, the membranes are formed of a same material as the ultrasound focusing lens.

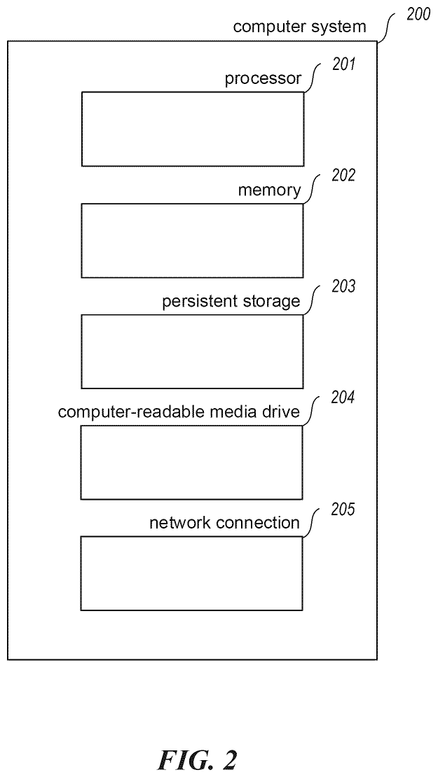

FIG. 2 is a block diagram showing some of the components typically incorporated in at least some of the computer systems and other devices on which the facility operates. In various embodiments, these computer systems and other devices 200 can include server computer systems, cloud computing platforms or virtual machines in other configurations, desktop computer systems, laptop computer systems, netbooks, mobile phones, personal digital assistants, televisions, cameras, automobile computers, electronic media players, physiological sensing devices, and/or their associated display devices, etc. In various embodiments, the computer systems and devices include zero or more of each of the following: a processor 201 for executing computer programs and/or training or applying machine learning models, such as a CPU, GPU, TPU, NNP, FPGA, or ASIC; a computer memory 202 for storing programs and data while they are being used, including the facility and associated data, an operating system including a kernel, and device drivers; a persistent storage device 203, such as a hard drive or flash drive for persistently storing programs and data; a computer-readable media drive 204, such as a floppy, CD-ROM, or DVD drive, for reading programs and data stored on a computer-readable medium; and a network connection 205 for connecting the computer system to other computer systems to send and/or receive data, such as via the Internet or another network and its networking hardware, such as switches, routers, repeaters, electrical cables and optical fibers, light emitters and receivers, radio transmitters and receivers, and the like. While computer systems configured as described above are typically used to support the operation of the facility, those skilled in the art will appreciate that the facility may be implemented using devices of various types and configurations, and having various components.

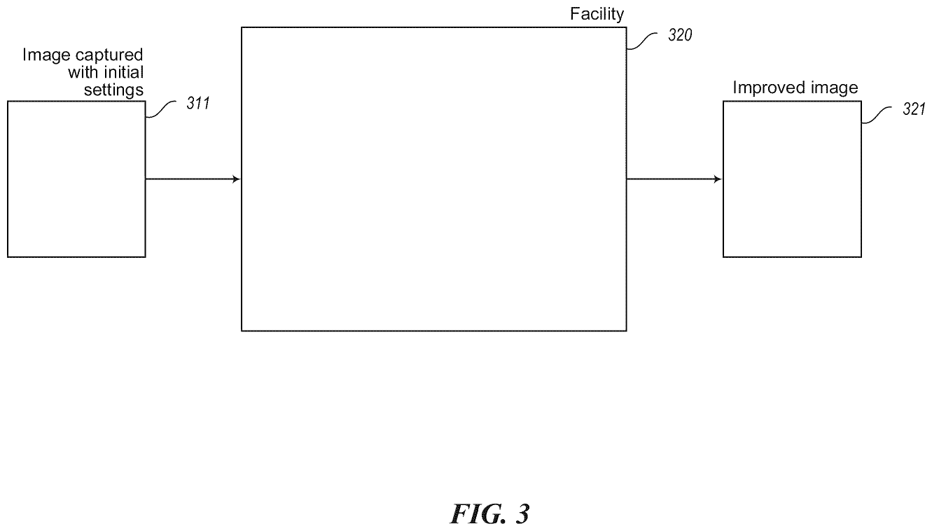

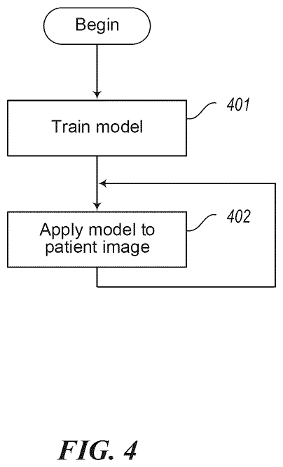

FIGS. 3 and 4 provide a generic view of the facility that spans many of its embodiments. FIG. 3 is a general data flow diagram showing the operation of the facility. In the diagram 300, the facility 320 receives an ultrasound image 311 captured with initial settings. By processing image 311, the facility produces an improved image 321 that typically is more usable for diagnostic and other analytical purposes than image 311.

FIG. 4 is a general flow diagram showing the operation of the facility with respect to one or more machine learning models used by the facility. In act 401, the facility uses training data to train a model, as discussed in further detail below with respect to particular groups of embodiments. In act 402, the facility applies the model trained in act 401 to patient images in order to achieve improved images like improved image 321. After act 402, the facility continues in 402 to apply the model to additional patient images.

Those skilled in the art will appreciate that the acts shown in FIG. 4 and in each of the flow diagrams discussed below may be altered in a variety of ways. For example, the order of the acts may be rearranged; some acts may be performed in parallel; shown acts may be omitted, or other acts may be included; a shown act may be divided into subacts, or multiple shown acts may be combined into a single act, etc.

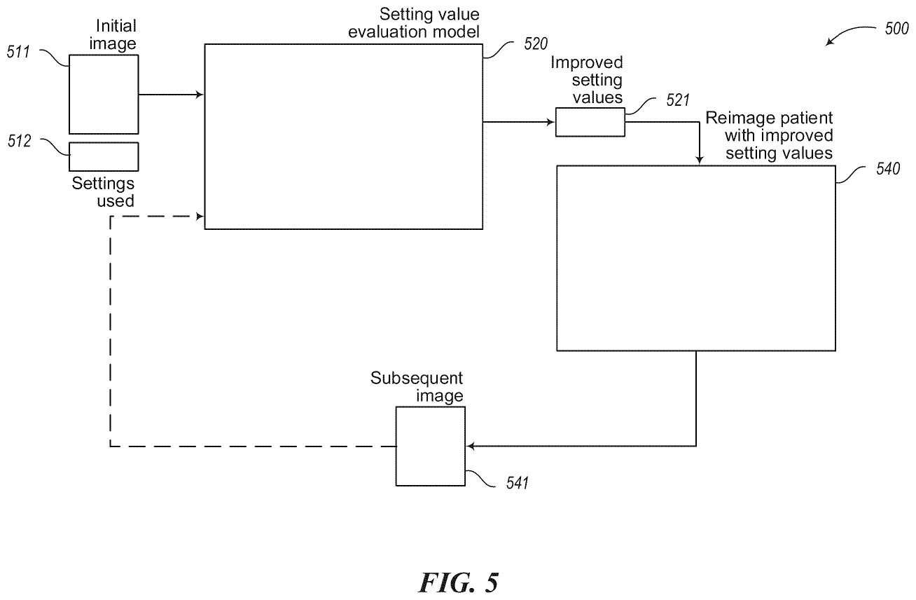

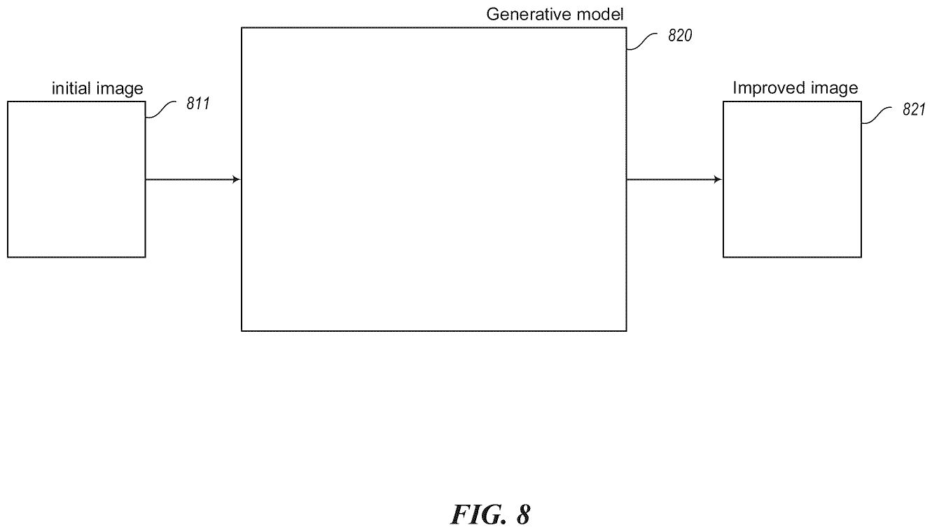

FIGS. 5-7 described below relate to setting improvement embodiments in which the facility applies machine learning techniques to images captured with certain settings to identify changes to the settings that would further optimize them, then causes reimaging using these improved settings. FIGS. 8-10 relate to generative embodiments in which the facility applies machine learning techniques to directly generate improved-quality images based upon images captured with suboptimal settings.

FIG. 5 is a data flow diagram showing a process performed by the facility in some of the setting improvement embodiments. In the diagram 500, the facility applies a setting value evaluation model 520 to an initial image 511 to predict a set of improved, more optimal settings 521 for capturing this image relative to the settings 512 actually used to capture this image. The facility then reimages a patient 540 with the improved setting values 521 to produce a subsequent image 541. In some embodiments, subsequent image 541 is used for diagnostic or other analytic purposes, and/or stored on behalf of the patient. In some embodiments, the facility performs one or more additional setting improvement cycles by applying the setting value evaluation model to one or more of the subsequent images.

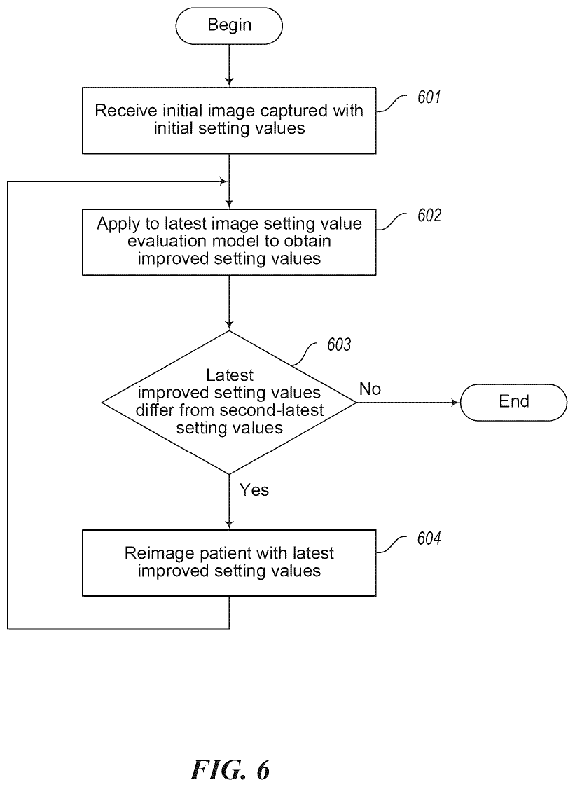

FIG. 6 is a flow diagram showing a process performed by the facility in some of the setting improvement embodiments. In act 601, the facility receives an initial image captured with initial setting values, e.g., initial image 511 captured with initial setting values 512. In act 602, the facility applies to the most recently-captured image a setting value evaluation model 520 to obtain improved setting values 521. In act 603, if the setting values obtained by the most recent iteration of act 602 differ from those obtained in the second-latest iteration of act 602, then the facility continues in act 604, else this process completes. In act 604, the facility reimages the patient with the improved setting values obtained in the most recent iteration of act 602. After act 604, the facility continues in act 602 to apply the model to the image captured in the most recent iteration of act 604.

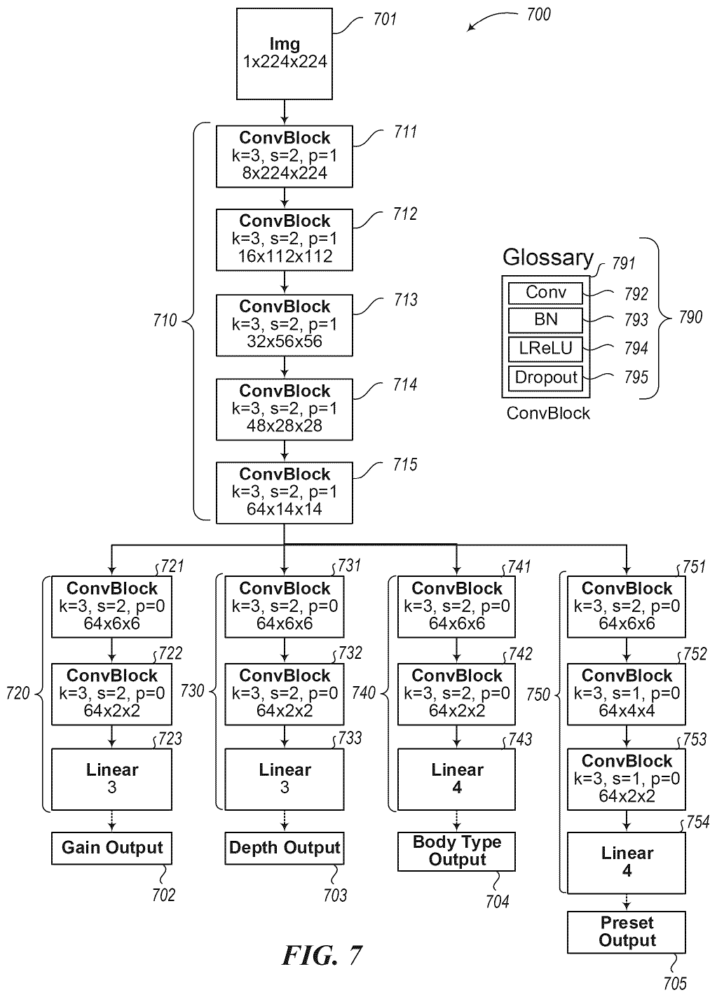

FIG. 7 is a model architecture diagram showing the organization of a machine learning model used by the facility in some of the setting improvement embodiments. A key or “glossary” 790 shows the composition of the ConvBlock structures shown in the architecture diagram 700. In particular, the glossary shows that a ConvBlock 791 is made up of a convolutional layer 792—such as a 2D convolutional layer, a batch normalization layer 793—such as a 2D batch normalization layer, a leaky ReLU activation function layer 794, and a dropout layer 795. The network includes convolutional blocks 711-715, 721-722, 731-732, 741-742, and 751-753, specifying for each a kernel size k, a stride s, a padding output shape p, and dimensions (channel×width×height). For example, the drawing shows that ConvBlock 711 has kernel size 3, stride 2, padding output shape 1, and dimensions 8×224×224. In addition to its conditional blocks, the network includes linear layers 723, 733, 743, and 754.

The network takes as its input an ultrasound image 701, such as a 1×224×224 grey scale ultrasound image. The network produces four outputs: a gain output 702 that predicts whether the gain setting value used to capture image 701 was too low, optimal, or too high; a depth output 703 that predicts whether the depth setting value used to capture image 701 was too shallow, optimal, or too deep; a body type output 704 that predicts whether the patient's body type is small, medium, or large; and a preset output 705 that predicts the region or region type of the body that was imaged, or other imaging scenario, such as heart, lungs, abdomen, or musculoskeletal. The output of branch 710 issuing from ConvBlock 715 is shared by branch 720 to produce the gain output, branch 730 to produce the depth output, branch 740 to produce the body type output, and branch 750 to produce the preset output.

Those skilled in the art will appreciate that a variety of neural network types and particular architectures may be straightforwardly substituted for the architecture shown in FIG. 7, and in the additional architecture diagrams discussed below.

FIG. 8 is data flow diagram showing a process performed by the facility in some of the generative embodiments. In the diagram 800, the facility applies a generative model 820 to an initial image 811 to predict an improved image 821, predicted by the generative model to be the result of recapturing the initial image with more optimal setting values.

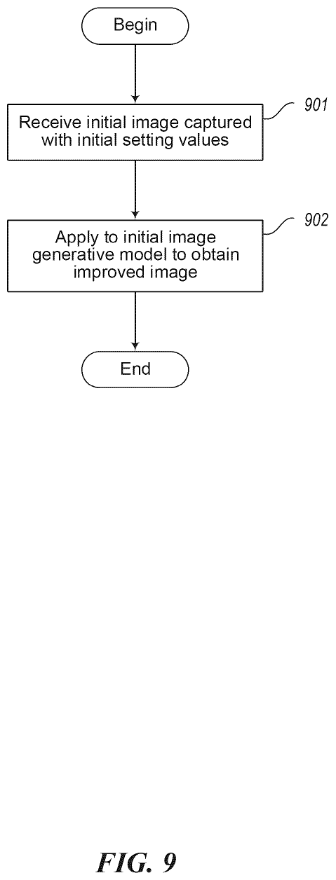

FIG. 9 is a flow diagram showing a process performed by the facility in some of the generative embodiments. In act 901, the facility receives an initial image captured with initial setting values, such as initial image 811. In act 902, the facilities applies to the initial image a generative model—such as generative model 820—to obtain an improved image, such as improved image 821. After act 902, this process concludes.

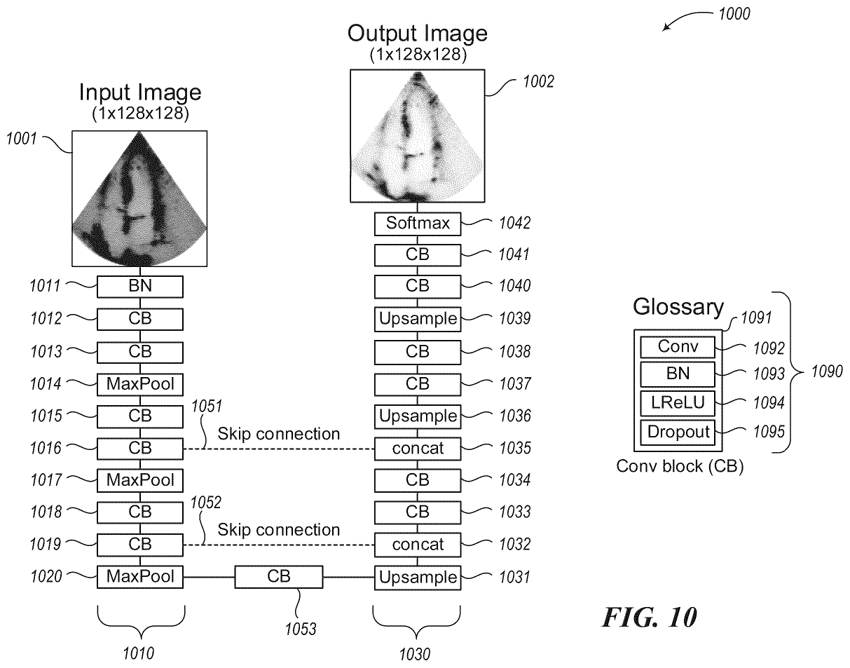

FIG. 10 is a model architecture diagram showing the organization of machine learning model used by the facility in some of the generative embodiments. In various embodiments, the facility uses a generative machine learning model that is a conditional generative adversarial deep learning network, or a residual U-net of another type. A glossary 1090 similar to glossary 790 shown in FIG. 7 shows the composition of the convolutional block structures shown in the architecture diagram 1000. In addition to the convolutional blocks (“CBs”) 1012, 1013, 1015, 1016, 1018, 1019, 1033, 1034, 1037, 1038, 1040, 1041, and 1053, the network includes batch normalization (“BN”) layer 1011; max pooling (“MaxPool”) layers 1014, 1017, and 1020; upsample layers 1031, 1036, and 1039; concatenation (“concat”) layers 1032 and 1035; and softmax activation function layer 1042. At a coarser level, the network is made up of a contracting path 1010 that performs encoding, and an expansive path 1030 that performs decoding. These two paths are joined by convolutional block 1053, as well as two skip connections 1051 and 1052. The network takes as its input an input image 1001 captured by an ultrasound machine using a set of initial setting values, and outputs an output image 1002 that predicts the contents of the input image had it been captured with setting values that were more optimal.

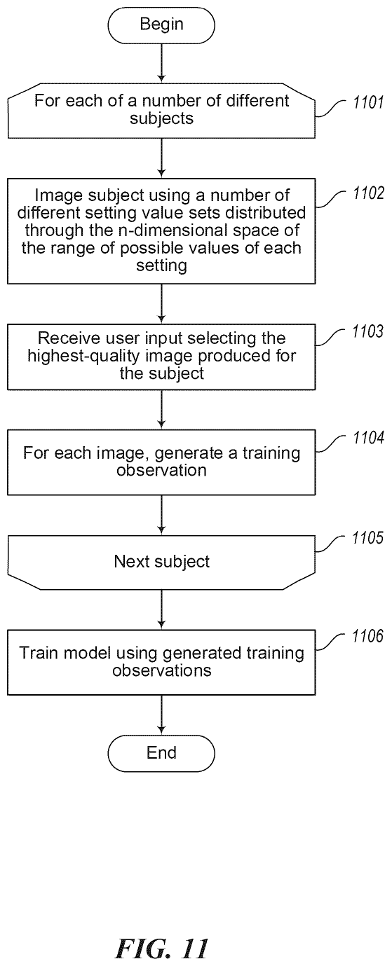

FIG. 11 is a flow diagram showing a process performed by the facility in some embodiments in order to train a machine learning model used by the facility, either a setting improvement model or a generative model. In acts 1101-1105, the facility loops through each of a number of different animal subjects, such as human subjects. In act 1102, the facility uses an ultrasound machine to image the current subject a number of times, each time using a different set of setting values. In particular, in some embodiments, these setting value sets are distributed in a fairly uniform manner across an n-dimensional region in which each dimension corresponds to the range of possible values for a different one of the ultrasound machine's settings. In act 1103, the facility presents the images captured for this subject, and receives user input from a human expert that selects from among them the highest-quality image produced for the subject. In act 1104, for each of the captured images, the facility generates a training observation. This step is discussed in detail below for each of the different model types. In act 1105, if additional subjects remain to be processed, the facility continues in act 1101 to process the next subject, else the facility continues to 1106. In act 1106, the facility trains its machine learning model using the training observations generated in act 1104. After act 1106, this process concludes, making the trained machine learning model available for application by the facility to patients.

For the setting value evaluation model, the facility generates a training observation in act 1104 as follows: for each setting, the facility uses the setting value used to capture the image to the setting value used to capture the image identified as the highest-quality image produced for the subject. The facility then establishes a training observation for the image in which the independent variable is the image, and the dependent variables are, for each setting, the result of the comparison of the value used for that setting to capture the image to the value used for that setting to capture the highest-quality image produced for the subject. For example, if the value of the depth setting used to capture the image was 9 cm and the value of the depth setting used to capture the highest-quality image produced for the subject was 11 cm, then the facility would use a “depth too shallow” value for one of the dependent variables in this observation. In some embodiments, for some settings, the facility simply uses the value of the setting used to capture the highest-quality image produced for the subject, without comparison to the corresponding value of the setting used to capture the image; for example, in such embodiments, where the value “large” is used for a body type setting to capture the highest-quality image produced for the subject, the facility uses this “large” setting value as a dependent variable for each of the observations produced from the images captured from the same subject.

For the generative model, the facility generates a training observation for each image in act 1104 as follows: the facility uses the image as the independent variable, and the highest-quality image produced for the same subject as the dependent variable.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A system, comprising:
an ultrasound machine having an ultrasound transducer; and
a computing device, the computing device comprising:
a communication interface configured to directly receive from the ultrasound machine ultrasound echo data sensed by the ultrasound transducer from a person, the received ultrasound echo data being sensed subject to an initial value of each of a plurality of ultrasound machine settings and comprising an initial ultrasound image; and
a processor configured to perform a method, the method comprising:
receiving the initial ultrasound image;
accessing a machine learning model trained to predict, from an ultrasound image captured subject to selected values of each of the plurality of ultrasound machine settings, optimal values for each of the plurality of ultrasound machine settings;
subjecting the initial ultrasound image to the machine learning model to obtain, for each of the plurality of ultrasound machine settings, a predicted optimal value for use in imaging the person; and
causing the ultrasound machine to reimage the person using the obtained predicted optimal values of the plurality of ultrasound machine settings.

2. The system of claim 1, the method further comprising:
receiving a second ultrasound image sensed by the ultrasound transducer from the person subject to the obtained predicted optimal values of the plurality of ultrasound machine settings; and
causing the received second ultrasound image to be persistently saved on behalf of the person.

3. The system of claim 1, the method further comprising:
receiving a second ultrasound image sensed by the ultrasound transducer from the person subject to the obtained predicted optimal values of the plurality of ultrasound machine settings; and
performing automatic medical diagnosis on the basis of the received second ultrasound image.

4. The system of claim 1 wherein the machine learning model is a classifying neural network.

5. The system of claim 1 wherein the plurality of ultrasound machine settings comprise:
depth,
gain,
time-gain-compensation,
body type,
imaging scenario, or
body region.

6. The system of claim 1, the method further comprising:
using ultrasound images captured from human subjects to train the machine learning model.

7. A method in a computing system, comprising:
receiving a ultrasound image visualizing part of a person's body captured by an ultrasound machine, the ultrasound machine having a plurality of settings each having a set of possible values, the ultrasound image's capture by the ultrasound machine reflecting, for each setting of the plurality of settings, a selected one of the setting's set of possible values; and
subjecting the received ultrasound image to a machine learning model trained using a plurality of training observations to obtain a resulting ultrasound image, each training observation of the plurality of training observations having (1) an independent variable that is a first ultrasound image captured by an ultrasound machine from a particular site of a subject's body using, for each setting of the plurality of settings, a first value among the setting's set of possible values, and (2) a dependent variable that is a second ultrasound image captured from the same site of the same subject's body using, for each setting of the plurality of settings, a second value among the setting's set of possible values, one or more of the second setting values being different from the corresponding first setting values, the second ultrasound image having been judged to better visualize the site of the subject's body than the first ultrasound image.

8. The method of claim 7 wherein the machine learning model is a residual U-net.

9. The method of claim 7 wherein the machine learning model is a conditional generative adversarial deep learning network.

10. The method of claim 7, further comprising using the plurality of training observations to train the machine learning model.

11. The method of claim 7, further comprising:
causing the obtained resulting ultrasound image to be persistently saved on behalf of the person.

12. The method of claim 7, further comprising:
performing automatic medical diagnosis on the basis of the obtained resulting ultrasound image.

13. One or more memory devices collectively storing a training observation data structure relating to an ultrasound machine configured to capture ultrasound images each on the basis of establishing a value for each of a plurality of ultrasound machine settings that is among a plurality of possible values for that setting, the data structure comprising:
a plurality of training observations, each training observation comprising:
an independent variable that is a first ultrasound image captured by an ultrasound machine from a particular site of a subject's body using, for each setting of the plurality of settings, a first value among the setting's set of possible values; and
a dependent variable that is based on a second ultrasound image captured from the same site of the same subject's body using, for each setting of the plurality of settings, a second value among the setting's set of possible values, one or more of the second setting values being different from the corresponding first setting values, the second ultrasound image having been judged to better visualize the site of the subject's body than the first ultrasound image,
such that the contents of the data structure are usable to train a machine learning model in turn usable to improve the quality of an ultrasound image captured using a set of ultrasound machine setting values that is collectively sub-optimal.

14. The one or more memory devices of claim 13 wherein the plurality of ultrasound machine settings comprise:
depth,
gain,
time-gain-compensation,
body type,
imaging scenario, or
body region.

15. The one or more memory devices of claim 14 wherein the second setting value sets of the plurality of training observations are well-distributed throughout an n-dimensional space defined by the possible values of each of the settings as a separate dimension.

16. The one or more memory devices of claim 14 wherein the optimal value of each of a first subset of the plurality of settings varies between subjects, and is consistent among imaging studies of the same subject,
and wherein, for each permutation of the possible values of the first subset of settings, the plurality of training observations comprise at least one training observation whose second setting values match the permutation.

17. The one or more memory devices of claim 14 wherein the optimal value of each of a first subset of the plurality of settings varies between imaging sites for the same subject,
and wherein the plurality of training observations comprise, for each of a plurality of subjects, for each of a plurality of imaging sites, at least one training observation from the imaging site of the subject.

18. The one or more memory devices of claim 14 wherein the optimal value of each of a first subset of the plurality of settings varies between sites for the same patient,
and wherein, for each permutation of the possible values of the first subset of settings, the plurality of training observations comprise at least one training observation whose second setting values match the permutation.

19. The one or more memory devices of claim 14 wherein, for each training observation, the dependent variable comprises at least one of the second setting values.

20. The one or more memory devices of claim 14 wherein, for each training observation, the dependent variable comprises, for each of at least a portion of the plurality of settings, results of comparing the first value for the setting with the second value for the setting.

21. The one or more memory devices of claim 14 wherein, for each training observation, the dependent variable comprises the second ultrasound image.

22. One or more memory devices collectively having contents configured to cause a computing system to perform a method relating to an ultrasound machine configured to capture ultrasound images each on the basis of establishing a value for each of a plurality of ultrasound machine settings that is among a plurality of possible values for that setting, the method comprising:
accessing a plurality of training observations, each training observation of the plurality of training observations having (1) an independent variable that is a first ultrasound image captured by an ultrasound machine from a particular site of a subject's body using, for each setting of the plurality of settings, a first value among the setting's set of possible values, and (2) a dependent variable that is a second ultrasound image captured from the same site of the same subject's body using, for each setting of the plurality of settings, a second value among the setting's set of possible values, one or more of the second setting values being different from the corresponding first setting values, the second ultrasound image having been judged to better visualize the site of the subject's body than the first ultrasound image; and
using the plurality of training observations to train a machine learning model.

23. The one or more memory devices of claim 22, the method further comprising:
persistently storing the trained state of the machine learning model.

24. The one or more memory devices of claim 22, the method further comprising:
applying the trained model to an ultrasound image captured from the patient.

25. The one or more memory devices of claim 22 wherein, for each training observation, the dependent variable comprises at least one of the second setting values.

26. The one or more memory devices of claim 22 wherein, for each training observation, the dependent variable comprises, for each of at least a portion of the plurality of settings, results of comparing the first value for the setting with the second value for the setting.

27. The one or more memory devices of claim 22 wherein, for each training observation, the dependent variable comprises the second ultrasound image.

28. One or more memory devices collectively storing a trained machine learning model data structure relating to an ultrasound machine configured to capture ultrasound images each on the basis of establishing a value for each of a plurality of ultrasound machine settings that is among a plurality of possible values for that setting, the data structure comprising:
state information produced by training a machine learning model to predict, based on an ultrasound image captured from a person using a first set of setting values, a different set of setting values that will produce a higher-quality ultrasound image if used to reimage the person,
wherein the model is usable to transform an input ultrasound image visualizing a imaging site of a person captured using a first set of setting values into a more-optimal set of setting values for use in reimaging the person.

29. The one or more memory devices of claim 28 wherein the trained machine learning model is a classifying neural network.

30. One or more memory devices collectively storing a training observation data structure relating to an ultrasound machine configured to capture ultrasound images each on the basis of establishing a value for each of a plurality of ultrasound machine settings that is among a plurality of possible values for that setting, the data structure comprising:
state information produced by training a machine learning model to predict, based on an ultrasound image captured from a person using a first set of setting values, a version of the ultrasound image transformed to correspond to setting values that is more optimal than the first set of setting values,
wherein the model is usable to transform an input ultrasound image visualizing a imaging site of a person captured using a first set of setting values into an output ultrasound image corresponding to a set of setting values that is more optimal than the first set of setting values.

31. The one or more memory devices of claim 30 wherein the trained machine learning model is a residual U-net.

32. The one or more memory devices of claim 30 wherein the trained machine learning model is a conditional generative adversarial deep learning network.
